SEQUENCE LISTING (7 sequences)

SEQ ID NO: 1 - DNA, 4873 bp, synthetic construct
  misc_feature 1..4873: note = Synthetic
  source 1..4873: mol_type = other DNA
aacaagaacaggggaaatcctagggctgacattgccagtggaaacatacaggctggagctctttagtcaggagctccagctgtgatctagacatcaggcaggaagatcaaatctgtcccaacaatacaggggacagaggctcaacctagagtgtgagcatcaggggctgtgcaggagatttcagagctcaggtgcagcagagactagcatggccctggggataaagggaaggatccaagggacaaggggataatcctggggaggtaagggccagcttcgtgacagaaggtggtggtgtccaacttcaagagccctgtgctacaatttaaaaaaaaaaaaaaaggaaagggacttctctgtgtttggcaacacaagtgcgatgcacaggcaggaagatcaaatctgtcccaacaatacaggggacagagggtcaacctacaaaaggaaagaacctggggcagtgtgaagacaacactgtagaagccaaggctgagttcactgagctctcgttagtgagactacacagcaaggaggtggcgggcactgagcagtgaggccccgggaagtgggggtgatggtggtgacggtggtaactgttaagaactgggggaaagaattgtggagaaccaagctaaatagttatgtcaaaccacatgtttaggagcctgggttgacttcatagggagtaggcatggaggctaatctagaggtttgtgtataggcaagaagtgaatcctgacccaagaatagagagtgctaaacggacttagttcaaagacaactgaaaaagacaatgcctgcaaaacaaagctaaggccagagctcttggactatgaagagttcagggaacctaagaacagggaccatctgtgtacaggccaaggccggtagaagcagcctaggaagtgtcaagagccaacgtggctgggtgggcaaagacaggaagggactgttaggctgcagggatgtgccgacttcaatgtgcttcagtattgtccagattgtgtgcagccatatggcccaggtataagaggtttaacagtggaacacagatgcccacatcagacagctggggggcgggggtgaacacagatacccatactggaaagcaggtggggcattttcctaggaacgggactgggctcaatggcctcaggtctcatctggtctggtgatcctgacattgataggcccaaatgttggatatcacctactccatgtagagagtcggggacatgggaagggtgcaaaagagcggccttctagaaggtttggtcctgtcctgtcctgtctgacagtgtaatcacatatactttttcttgtagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattggtgagaggacgtatagggaggaggggttcactagaggtgaggctcaagccattagcctgcctaaaccaaccaggctggacagccatcaccaggaaatggatctcagcccagaagatcgaaagttgttcttctcccttctggagatttctatgtcctttacactcattggttaatatcctgggttggattcccacacatcttgacaaacagagacaattgagtatcaccagccaaaagtcatacccaaaaacagcctggcatgacctcacaccagactcaaacttaccctacctttatcctggtggcttctcatctccagaccccagtaacacatagctttctctccacagtgcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctcctggtaaatgatcccagtgtccttggagccctctggtcctacaggactctgacacctacctccacccctccctgtgtaaataaagcacccagcactgccttgggaccctgcaataatgtcctggtgatttctgagatgtagagtctagctaggtcatggaatgaggggtctccatggtttgaggcctgagttgtgactaaggaaaaacccataggcctacactgccacacccagcacttttgaatttgcctgacatgaaaagaatttacctctccctggaaagtggagccttatccctaggcagttcccttaccagaccttcctctagcttgcactttgttctgggcacagaatgtgtctaaccccccaaagcaaggaagacacaacctctacctccctcactctgtccttaccccttttcctggctaagcatctcactgagtgcgctgaatagatgcatgtggccacagtcttgcagacagacccttgccatctctccactcagctttccagaggctaagtctagcccgtatggtgataatgcagggagctctatgctatctcagtgctatcagactcccaagtggaggatgaacatggacccattaaaaccaacctgcgcagcaacaccctgccaataaggcccgtatgtgaaaatgtgcacacatctacacatgcacaggcacacacacacacacatgcatgggcacacacacatacagagagagagaatcacagaaactcccatgagcatcctatacagtactcaaagataaaaaggtaccaggtctacccacatgatcatcctcggcatttacaagtgggccaactgatacagataaaacttttctatgccaaggacgccaacatatacacaagtccgctcatgacaaatctgtccctgaacctcagactggcgcccgtgactcatacagtggacactcctccaaagctgtatagcttcctttacttccctgtgtgtactttctctgaagtacactcatcacacagaagaggccctgtgattactctggccctctgttcttggtcatcagagaatagacagaagatcaggcaaactacacagacacttcccacaatcatcacaggccctgactctgctctccagtctcaaaactgaaggctggagcacacagaataagctcctgcacaggccaggccagtatcgggtccagtgtgtctgactgagcccagggacaaaatggcagcactttggggaactgaggtttctggtccaagaaggagagatggaggcccagggagggtctgctgacccagcccagcccagcccagctgcagctttctcctgggcctccatacagcctcctgccacacagggaatggccctagccccaccttattgggacaaacactgaccgccctctctgtccagggctgcaactggacgagacctgtgctgaggcccaggacggggagctggacgggctctggacgaccatcaccatcttcatcagcctcttcctgctcagtgtgtgctacagcgctgctgtcacactcttcaaggtcagccatactgtccccacagtgtctacaatgtcctcatactcttccccatactgtccctgtggtgacctataccccacactgtcccatgctaatgaccacagtcttacatgctatgtaatgctgtctacccttctgtatgcacagtctcacaatgtcccatgcagtctccacgatgctccatactgtccccattccaacccatgctgccccttgttccccgctatgctgtcccatgctattgtctgtattttcatgctcttttcacactgtccctagtgtcacattctgcccatgttgtccaccacattgtccccactctgtacacagcctcacactgtaccctgctacccgataatgttccctgttgtccccaactctctccctgcaccatttgtcaactgtcccctgaattcccatgttgttcccacactgttagtgtgtaatgtgctctgtccca

SEQ ID NO: 2 - protein, 11 aa, Homo sapiens
PKSCDKTHTCP

SEQ ID NO: 3 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 22 bp, synthetic construct
  misc_feature 1..22: note = Synthetic
  source 1..22: mol_type = other DNA
gcacaatccctgggcactgtgg

SEQ ID NO: 5 - DNA, 21 bp, synthetic construct
  misc_feature 1..21: note = Synthetic
  source 1..21: mol_type = other DNA
gagcctctcccactctcctgg

SEQ ID NO: 6 - protein, 397 aa, synthetic construct
  REGION 1..397: note = Synthetic
KTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGLQLDETCAEAQDGELDGLWTTITIFISLFLLSVCYSAAVTLFKVKWIFSSVVELKQTLVPEYKNMIGQAP

SEQ ID NO: 7 - protein, 328 aa, synthetic construct
  REGION 1..328: note = Synthetic
KTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0049007 A1
(43) Pub. Date: Feb. 13, 2025

(54) HUMANIZED MOUSE MODELS OF THE FCRN RECYCLING PATHWAY

(71) Applicant: The Jackson Laboratory, Bar Harbor, ME (US)

(72) Inventors: Michael V. Wiles, Bar Harbor, ME (US); Derry C. Roopenian, Bar Harbor, ME (US); Gregory Christianson, Bar Harbor, ME (US); Benjamin E. Low, Bar Harbor, ME (US)

(73) Assignee: The Jackson Laboratory, Bar Harbor, ME (US)

(21) Appl. No.: 18/926,242

(22) Filed: Oct. 24, 2024

Related U.S. Application Data
(62) Division of application No. 17/680,379, filed on Feb. 25, 2022, now Pat. No. 12,156,517.
(60) Provisional application No. 63/154,500, filed on Feb. 26, 2021.

(51) Int. Cl. A01K 67/0278; C07K 14/735; C07K 16/28

(52) U.S. Cl. CPC A01K 67/0278; C07K 14/70535; C07K 16/283; A01K 2207/15; A01K 2227/105; C07K 2317/21; C07K 2317/24; C07K 2317/52; C07K 2317/72; C07K 2317/92; C07K 2317/94

(57) ABSTRACT

The present disclosure provides improved humanized IgG1 FCRN mouse models for use, for example, in estimating serum half-life of therapeutic proteins such as monoclonal antibodies.

### RELATED APPLICATIONS

This application is a divisional of U.S. application Ser. No. 17/680,379, filed Feb. 25, 2022, which claims the benefit under 35 U.S.C. § 119 (e) of U.S. provisional application No. 63/154,500, filed Feb. 26, 2021, each of which is herein incorporated by reference in its entirety.

### GOVERNMENT LICENSE RIGHTS

This invention was made with government support under OD011190 awarded by National Institutes of Health. The government has certain rights in the invention.

### REFERENCE TO AN ELECTRONIC SEQUENCE LISTING

The contents of the electronic sequence listing (J022770080US02-SEQ-HJD.xml; Size: 14,577 bytes; and Date of Creation: Oct. 23, 2024) is herein incorporated by reference in its entirety.

### BACKGROUND

The use of therapeutic proteins such as monoclonal antibodies are among the fastest growing and most promising advances in modern healthcare, leading a revolution in many therapeutic areas, including cancer, inflammatory disease, and diabetes. An important step toward bringing novel therapeutic proteins to market is obtaining an accurate estimate of the elimination half-life of the proteins. The elimination half-life of a therapeutic protein is a pharmacokinetic parameter that is defined as the time it takes for the concentration of the protein in the blood (e.g., serum) to be reduced by 50%. The elimination half-life is a useful pharmacokinetic parameter as it provides an accurate indication of the length of time that the effect of the therapeutic protein persists in an individual. It can also show if accumulation of the protein is likely to occur with a multiple dosing regimen. This is helpful when it comes to deciding the appropriate dose amount and frequency.

The elimination half-life of therapeutic proteins in an individual is regulated by the recycling molecule neonatal Fc receptor (FcRn). FcRn binds to the protein intracellularly to initiate the recycling pathway through which the protein is diverted away from the lysosomal degradation pathway and is instead transported in recycling transport vesicles to the cell surface, where it is released back into the serum as reviewed in Roopenian, D. C., et al. Nat. Rev. Immunol, 2007;7:715-725. The use of mouse models to replicate this pathway in human, to evaluate the in vivo stability of therapeutic antibodies and other Fc-based biologics for example, is an effective and proven alternative to the use of non-human primates (Valente, D., et al. MABS, 2020; 12 (1): 1829337, and Betts, A., et al. MABS, 2018;10 (5): 1462429).

### SUMMARY

A major asset of many monoclonal antibody (mAb)-based biologics is their persistence in circulation. The MHC class I family Fc receptor, FCGRT, is primarily responsible for this extended pharmacokinetic behavior. Engagement of FCGRT with the crystallizable fragment (Fc) domain protects IgG from catabolic elimination, thereby extending the persistence and bioavailability of IgG and related Fc-based biologics. There is a need for reliable in vivo models to facilitate the preclinical development of novel IgG-based biologics. FcRn-humanized mice have been widely accepted as translationally relevant surrogates for IgG-based biologics evaluations. Although such FCGRT-humanized mice, especially the mouse strain, B6.Cg-Fcgrttm1Dcr Tg (FCGRT)32Dcr (abbreviated Tg32; JAX Stock #014565), have been substantially validated for modeling humanized IgG-based biologics, there is a recognized caveat—they lack an endogenous source of human IgG that typifies the human competitive condition. Herein, CRISPR/Cas9-mediated homology-directed repair was used to equip the hFCGRT Tg32 strain with a human IGHG1 Fc domain. This replacement now results in mice that produce human IgG1 Fc-mouse IgG Fab2 chimeric antibodies at physiologically relevant levels, which can be further heightened by immunization. This endogenous chimeric IgG1 significantly dampens the serum half-life of administered humanized mAbs in an hFCGRT-dependent manner. Thus, such IgG1-Fc humanized mice may provide a more physiologically relevant competitive hFCGRT-humanized mouse model for the preclinical development of human IgG-based biologics. See Low, B. E., et al. MABS, January-December 2020;12 (1): 1829334, which is incorporated by reference herein in its entirety.

The current Tg32 mouse model carries a knock-out mutation for the mouse Fcgrt (Fc receptor, IgG, alpha chain transporter) gene and a transgene expressing the human FCGRT gene under the control of its own native promoter (hTg32). This Tg32 mouse model, while useful in evaluating the pharmacokinetics and pharmacodynamics of human immunoglobulin G (IgG) and Fc-domain based therapeutics, lacks IgG that can compete for hFcRn-mediated antibody recycling, due to the species-discrimination of human FcRn and the resulting failure to recycle and maintain physiological levels of mouse IgG. The lack of competing human IgG in the Tg32 mouse model limits its use in preclinical modeling of antibody recycling. There is no competition between an exogenous test human IgG (e.g., mAb) and endogenous mouse IgG for the hFcRn recycling pathway. This limitation means that the Tg32 mouse model and similar mouse models currently available only partially reflect normal human physiologically of antibody turnover, confounding pharmacokinetic (PK) studies for novel monoclonal antibody-based therapeutics, for example.

To overcome this limitation the improved humanized FcRn mouse models provided herein are genetically modified to include chimeric IgG1 that includes a fully human Fc region (˜0.9 kb region including the hinge, CH2 and CH3 domains) joined to a fully mouse Fab region with an intact mouse variable region. This chimeric IgG1, with its human IgG1 Fc region, can bind human FcRn (e.g., genomically encoded by a FCGRT transgene) to facilitate antibody recycling. Surprisingly, data provided herein shows that this mouse model, unlike other similar models, produces levels of serum IgG1, which rise following immunization with 2,4-Dinitrophenyl hapten conjugated to keyhole limpet hemocyanin (DNP-KLH). In particular, naïve Tg32-hFc mice yielded chimeric IgG1 levels that ranged from 280 to 1,320 μg/ml (mean 600 μg/ml) and following immunization of Tg32-hFc mice with 2,4-dinitrophenyl-keyhole limpet hemocyanin (DNP-KLH), chimeric IgG1 was significantly increased (mean value 4,200 μg/ml) (see FIG. 2a of Low, B. E., et al. MABS, January-December 2020; 12 (1): 1829334). This is well above level observed with the current mouse models. Further, data provided herein following treatment with two different monoclonal antibodies (1) HuLys11, a humanized monoclonal antibody (mAb) containing the CH1-3 region of human IgG1 that was raised against hen egg-white lysozyme, and (2) trastuzumab, a humanized mAb that binds the extracellular domain of the HER2 receptor and is in clinical use, demonstrates the occurrence of competition for human FcRn-meditated antibody recycling, closely reflecting normal physiologically function in humans. See Low, B. E., et al. (2020).

There is another beneficial outcome resulting from immunizing Tg32 mice expressing chimeric IgG1 with DNP-KLH: the demonstration of the utility of this mouse model to produce affinity matured chimeric IgG1 monoclonal antibodies that could act as therapeutic candidates. While the mg/ml serum concentrations of chimeric IgG1 became elevated following immunization, a large portion of the chimeric IgG1 activity was also demonstrated to be DNP-specific (FIG. 4). While DNP-specificity was demonstrated, any human therapeutic target could easily have been substituted yielding a chimeric IgG1 target-specific outcome.

Thus, some aspects of the present disclosure provide a mouse comprising a nucleic acid encoding human neonatal Fc receptor (FCRN) and a nucleic acid encoding a chimeric immunoglobulin that comprises a human immunoglobulin gamma (IgG) Fc region and a mouse Fab region, wherein the mouse comprises a null mutation in endogenous FcRn.

In some embodiments, the human IgG Fc region is a human IgG1 Fc region.

In some embodiments, cells of the mouse express the human FCRN and the chimeric immunoglobulin (B6.Cg-Fcgrt^{tm1Dcr} Ighg1^{em2(IGHG1)Mvw} Tg (FCGRT) 32Dcr/Mvw; also referred to as Tg32-hFc, JAX Stock #029686).

In some embodiments, a level of the chimeric immunoglobin in serum of the mouse is at least 280 μg/ml. For example, a level of the chimeric immunoglobin in serum of the naïve mouse may be 280 μg/ml to 1,320 μg/ml.

In some embodiments, the Fab region comprises a mouse constant light chain (mC_{L}) sequence and/or a mouse constant heavy chain 1 (mC_{H1}) sequence.

In some embodiments, the Fab region comprises a mouse variable light chain (mV_{L}) sequence and/or mouse variable heavy chain (mV_{H}) sequence.

In some embodiments, the human IgG1 Fc region comprises a human heavy chain 2 (hC_{H2}) sequence and/or a human heavy chain 3 (hC_{H3}) sequence.

In some embodiments, the human IgG1 Fc region comprises a human heavy chain sequence having at least 80%, at least 85%, at least 90%, at least 95%, or 100% identity to the amino acid sequence of SEQ ID NO: 3.

In some embodiments, the immunoglobulin comprises a human hinge sequence.

In some embodiments, the human hinge sequence comprises the amino acid sequence of SEQ ID NO: 2.

In some embodiments, the nucleic acid encoding the immunoglobulin comprises a sequence that has at least 70%, at least 75%, at least 80%, at least 85%, at least 90%, at least 95%, or 100% identity to the sequence of SEQ ID NO: 1.

In some embodiments, the human FcRn is encoded by human FCGRT (Fc receptor, IgG, alpha chain transporter).

In some embodiments, the chimeric immunoglobulin competes with exogenous IgG for binding to the human FcRn.

Also provided herein is a nucleic acid or set of nucleic acids encoding a chimeric immunoglobulin, wherein the chimeric immunoglobulin comprises a human immunoglobulin gamma (IgG) Fc region and a mouse Fab region.

Further provided herein is a chimeric immunoglobulin comprising a human IgG Fc region and a mouse Fab region.

Some aspects of the present disclosure provide a method of producing the mouse of any one of the preceding claims comprising modifying the genome of a mouse to express a nucleic acid or set of nucleic acids encoding a chimeric immunoglobulin, wherein the chimeric immunoglobulin comprises a human IgG Fc region and a mouse Fab region. In some embodiments, the mouse expresses human FCRN.

Other aspects of the present disclosure provide a method of producing a mouse comprising modifying the genome of a B6.Cg-Fcgrt^{tm1Dcr}Tg(FCGRT)32Dcr/DcrJ (Tg32) mouse to express a nucleic acid or set of nucleic acids encoding a chimeric immunoglobulin, wherein the chimeric immunoglobulin comprises a human IgG Fc region and a mouse Fab region.

Yet other aspects of the present disclosure provide a method comprising administering to the mouse a therapeutic antibody and assaying half-life of the therapeutic antibody in serum of the mouse. In some embodiments, the therapeutic antibody is a monoclonal IgG antibody. In some embodiments, the monoclonal IgG antibody is a monoclonal IgG1 antibody.

Still other aspects of the present disclosure provide a use of the mouse of any one of the preceding paragraphs to produce Fc-humanized IgG1 monoclonal antibodies.

### BRIEF DESCRIPTION OF THE DRAWINGS

FIG. 1 shows the structure of a chimeric IgG1 antibody of the present disclosure having a fully mouse Fab region and a fully human Fc region. Each heavy chain portion of the Fab region includes a mouse variable heavy chain domain (mV_{H}) and a mouse constant heavy chain domain 1 (mC_{H1}), and each light chain portion of the Fab region includes a mouse variable light chain domain (mV_{L}) and a mouse constant light chain domain (mC_{L}). Each heavy chain portion of the Fc region includes a human constant heavy chain domain 2 (hC_{H2}) and a human constant heavy chain domain 3 (hC_{H3}). The hinge region is depicted as double solid lines.

FIG. 2 shows the IgG1 domains in the mouse genome, the donor DNA encoding human IgG1, and the mouse genome after removal of the mouse hinge and Fc region (mC_{H2} and mC_{H3}) and insertion of the human hinge and Fc region (hC_{H2} and hC_{H3}). Transmembrane, TM. Predicted mRNAs from the region were verified by RT-PCR; note both isoforms were found and their correct splicing and sequence confirmed. Mouse sequence is shown in blue, human in green.

FIG. 3 shows the serum level of total human IgG1 Fc in milligrams per milliliter (mg/mL). “Tg32-hFc” is a mouse strain of the present disclosure expressing human FcRn and chimeric mouse/human IgG1 (see FIG. 1). “Tg32” is a mouse strain expressing human FcRn and endogenous mouse IgG1. Tg32-hFc, Immunized values are from a 1:160,000 serum dilution; Tg32-hFc, Non-Immunized values are from a 1:40,000 serum dilution; and Tg32, Non-Immunized values are from a 1:20,000 serum dilution. Immunized means that the mice are injected with 2,4-Dinitrophenyl hapten conjugated to keyhole limpet hemocyanin (DNP-KLH) and non-immunized means that the mice are not injected with DNP-KLH. The values plotted are average±SEM. See also Table 1 and FIG. 3 of Low, B. E., et al. (2020).

FIG. 4 shows the chimeric IgG1 anti-2,4-Dinitrophenyl (DNP) activity resulting following immunization of Tg32-hFc with DNP-KLH. Serum collected 2 weeks after the second immunization was diluted 1:1000, 1:5000, or 1:25000 then added to ELISA plates coated with DNP-bovine albumin, detected using mouse anti-human IgG1 Fc-alkaline phosphatase. Following incubation with substrate, OD405 was read. Unimmunized (naïve) Tg32-hFc serum showed no anti-DNP activity when diluted 1:1000. The values plotted are average±SEM. See also FIG. 2d of Low, B. E., et al. (2020).

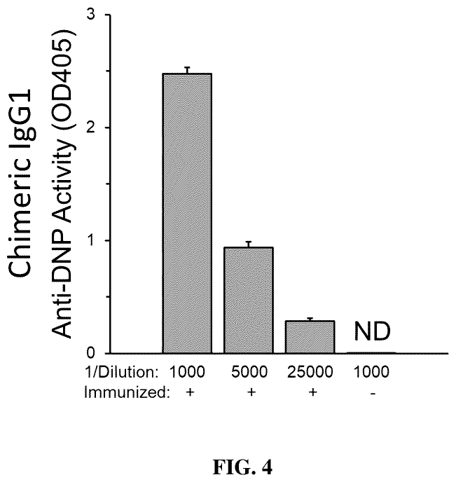

### DETAILED DESCRIPTION

The present disclosure provides, in some aspects, a mouse model that may be used to predict the half-life of candidate therapeutic antibodies (e.g., IgG antibodies) by modeling the recycling of human immunoglobulin gamma 1 (IgG1) antibodies. The neonatal Fc receptor (FcRn) rescues both albumin and IgG following endocytosis and thereby extends the half-life of these proteins. Exogenous IgG antibodies introduced into humans (and mice) compete with endogenous IgG1 for recycling, resulting in lower (e.g., shorter) serum half-lives of the antibodies. In order to accurately predict the half-life of a candidate therapeutic antibodies, for example, it is important that the mouse model genomically express physiologically-relevant levels of human FcRn and human or humanized IgG1. Mouse IgG1 cannot effectively compete with exogenous therapeutic antibodies for binding to human FcRn.

Thus, the development of the Tg32-hFc mice described herein provides a relevant and economic model to rapidly evaluate novel engineered Fc-based biologics within a more competitive environment, and could provide a more reliable preclinical PK analyses model. This Tg32-hFc model also provides a convenient vehicle for the rapid generation of Fc-humanized IgG1 monoclonal antibodies.

Thus, provided herein, in some aspects, are mouse models comprising a genomically-expressed chimeric immunoglobulin that competes with exogenous IgG for binding to human FcRn. Exemplary methods for assessing antibody competition for human FcRn-mediated IgG1 recycling is provided in Example 2.

### Human Neonatal Fc Receptor (FcRn) and IgG

The mouse models provided herein, in some embodiments, comprise a nucleic acid encoding a human neonatal Fc receptor (FcRn). FcRn, encoded by the Fogrt gene, is an MHC class I-like transmembrane protein that includes a heavy chain containing three extracellular domains (α1, α2 and α3), a single pass transmembrane domain and a short cytoplasmatic tail. For proper function, the FcRn heavy chain non-covalently associates with the common β2-microglobulin subunit as a light chain, which interacts with FcRn via residues on the underside of the α1-α2 platform and the side of the α3 domain. Intracellular binding of FcRn to an immunoglobulin such as IgG1 initiates the recycling pathway through which the IgG1 is diverted away from the lysosomal degradation pathway. The IgG is instead transported in recycling transport vesicles to the cell surface where it is released back into the serum.

The human FcRn expressed by cells of the mouse models provided herein, in some embodiments, is encoded by a transgene. In some embodiments, the transgene comprises the sequence of the human FCGRT genc (e.g., NCBI Gene ID: 2217). In some embodiments, the transgene comprises an hTg32 promoter.

The hFcRn, in some embodiments, is expressed in tissue of the mouse model at physiologically relevant levels.

The mouse models provided herein, in some embodiments, comprise a nucleic acid encoding a chimeric immunoglobulin, such as IgG. IgG molecules are approximately 150 kDa and composed of two identical light chains and heavy chains. IgG is the most abundant immunoglobulin in humans. It should be understood that “a nucleic acid encoding a chimeric immunoglobulin” encompasses a single nucleic acid or multiple nucleic acids encoding the heavy chains and light chains of the immunoglobulin.

A chimeric immunoglobulin is genetically engineered, for example, by joining the regions of a mouse immunoglobulin to regions of a human immunoglobulin. The chimeric IgG provided herein includes a human Fc region (e.g., including a hinge region, a hC_{H2} domain, a hC_{H3} domain) and a mouse Fab region (e.g., including a mV_{H}, mV_{L}, mC_{H1}, and m_{CL}). The fragment crystallizable (Fc) region is the tail region of an immunoglobulin that interacts with cell surface receptors referred to as Fc receptors and some proteins of the complement system to activate the immune system. The Fc region of IgG, for example, includes two identical protein fragments, derived from the second and third constant domains of the two heavy chains. The antigen-binding fragment (Fab) is a region on an immunoglobulin that binds to antigens. It includes a constant domain and a variable domain of each of the heavy and the light chain. The variable domain contains the paratope (the antigen-binding site), comprising a set of complementarity-determining regions, at the amino terminal end of the monomer. Each arm of the Y thus binds an epitope on the antigen.

In some embodiments, the human IgG1 Fc region comprises a human heavy chain sequence having at least 80% identity to the amino acid sequence of SEQ ID NO: 3. For example, the human IgG1 Fc region may comprise a human heavy chain sequence having at least 85%, at least 90%, at least 95%, or 100% identity to the amino acid sequence of SEQ ID NO: 3.

The hinge region of an immunoglobulin is a flexible amino acid stretch in the central part of the heavy chains of the immunoglobulin that links these two chains by disulfide bonds. In some embodiments, an immunoglobulin comprises a human hinge sequence. In some embodiments, the human hinge sequence comprises the amino acid sequence of SEQ ID NO: 2.

In some embodiments, the immunoglobulin comprises a heavy chain sequence that has at least 80%, at least 85%, at least 90%, at least 95%, or 100% identity to the amino acid sequence of SEQ ID NO: 6 or SEQ ID NO: 7.

In some embodiments, the nucleic acid encoding the immunoglobulin comprises a sequence that has at least 80%, at least 85%, at least 90%, at least 95%, or 100% identity to the amino acid sequence of SEQ ID NO: 1.

In some embodiments, a level of the chimeric immunoglobin in serum of the mouse is at least 280 μg/ml. For example, the chimeric immunoglobulin may be expressed at a level of 3.0 mg/ml, 3.5 mg/ml, 4.0 mg/ml, 4.5 mg/ml, 5.0 mg/ml, 5.5 mg/ml, 6.0 mg/ml, 6.5 mg/ml, 7.0 mg/ml, 7.5 mg/ml, 8.0 mg/ml, 8.5 mg/ml, 9.0 mg/ml, 9.5 mg/ml, 10.0 mg/ml, 10.5 mg/ml, 11.0 mg/ml, 11.5 mg/ml, or 12.0 mg/ml. In some embodiments, a level of the chimeric immunoglobin in serum of the mouse is 3.0 mg/ml to 12 mg/ml, 3.5 mg/ml to 12 mg/ml, 4.0 mg/ml to 12 mg/ml, 4.5 mg/ml to 12 mg/ml, 5.0 mg/ml to 12 mg/ml, 5.5 mg/ml to 12 mg/ml, 6.0 mg/ml to 12 mg/ml, 6.5 mg/ml to 12 mg/ml, 7.0 mg/ml to 12 mg/ml, 7.5 mg/ml to 12 mg/ml, 8.0 mg/ml to 12 mg/ml, 8.5 mg/ml to 12 mg/ml, 9.0 mg/ml to 12 mg/ml, 9.5 mg/ml to 12 mg/ml, 10.0 mg/ml to 12 mg/ml, or 10.5 mg/ml to 12 mg/ml.

### Mouse Models

The mouse models of the present disclosure are based on the mouse model having the genotype B6.Cg-Fcgrt^{tm1Dcr} Tg (FCGRT)32Dcr/DcrJ (also referred to Tg32, JAX Stock #014565). Tg32 mice carry a knock-out mutation for the mouse Fegrt (Fc receptor, IgG, alpha chain transporter) gene and a transgene expressing the human FCGRT gene under the control of its own native promoter (hTg32). The genome of the Tg32 mouse model, however, encodes endogenous mouse IgG1 and thus these mice are limited with respect to their use in assessing the FcRn recycling pathway—mouse IgG1 cannot compete with exogenous immunoglobulins (e.g., IgG antibodies) for binding to FcRn.

The mouse models provided herein address this limitation by providing mice that instead express a chimeric form of IgG1 (B6.Cg-Fcgrt^{tm1Dcr} Ighg1^{em2(IGHG1)Mvw} Tg(FCGRT)32Dcr/Mvw; also referred to as Tg32-hFc, JAX Stock #029686), which is unexpectedly expressed at levels comparable to human IgG1 expression levels. These chimeric IgG1 expression levels are thus physiologically relevant and can be further heightened by immunization with antibodies (e.g., humanized monoclonal antibodies). Additionally, these chimeric IgG1 antibodies compete with exogenous immunoglobulins for binding to endogenous FcRn.

Herein, for simplicity, reference is made to “mouse” and “mouse models” (e.g., surrogates for human conditions). It should be understood that these terms, unless otherwise stated, may be used interchangeably throughout the specification to encompass “rodent” and “rodent models,” including mouse, rat and other rodent species.

It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The registry is maintained at the Institute for Laboratory Animal Research (ILAR) at the National Academy of Sciences, Washington, D.C. Lab Codes may be obtained electronically at ILAR's web site (nas.edu/cls/ilarhome.nsf). See also Davisson MT, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

The mouse models provide herein are transgenic mouse models that express human FCRN and a chimeric mouse/human IgG antibody, and do not express mouse FcRn. A transgenic mouse is a mouse having an exogenous nucleic acid (e.g., transgene) in (integrated into) its genome. Methods of producing transgenic mice are well-known.

Three conventional methods used for the production of transgenic mice include DNA microinjection (Gordon and Ruddle, Science 1981:214: 1244-124, incorporated herein by reference), embryonic stem cell-mediated gene transfer (Gossler et al., Proc. Natl. Acad. Sci. 1986, 83:9065-9069, incorporated herein by reference) and retrovirus-mediated gene transfer (Jaenisch, Proc. Natl. Acad. Sci. 1976, 73:1260-1264, incorporated herein by reference), any of which may be used as provided herein. Genomic editing methods using, for example, clustered regularly interspace palindromic repeats (CRISPR/Cas) nucleases, transcription activator-like effector nucleases (TALENs), or zinc finger nucleases (ZFNs) are described elsewhere herein.

Following delivery of nucleic acids to a fertilized embryo (e.g., a single-cell embryo (e.g., a zygote) or a multi-cell embryo (e.g., a developmental stage following a zygote, such as a blastocyst), the fertilized embryo is transferred to a pseudopregnant female, which subsequently gives birth to offspring. The presence or absence of a nucleic acid encoding human FcRn and/or a chimeric IgG antibody may be confirmed, for example, using any number of genotyping methods (e.g., sequencing and/or genomic PCR).

### Immunodeficient Mouse Models

Provided herein, in some embodiments, are immunodeficient mouse models. As is known in the art, immunodeficient mice have impaired or disrupted immune systems, such as specific deficiencies in MHC class I, II or both, B cell or T cell defects, or defects in both, as well as immunodeficiency due to knockdown of genes for cytokines, cytokine receptors, TLR receptors and a variety of transducers and transcription factors of signaling pathways. Immunodeficiency mouse models include the single-gene mutation models such as nude-mice (nu) strains and the severe combined immunodeficiency (scid) strains, non-obese diabetic (NOD) strain, RAG (recombination activating gene) strains with targeted gene deletion and a variety of hybrids originated by crossing doubly and triple mutation mice strains with additional defects in innate and adaptive immunity.

Non-limiting examples of spontaneous and transgenic immunodeficient mouse models include the following mouse strains:
Nude (nu) [Flanagan SP. Genet Res 1966; 8:295-309; and Nehls M et al. Nature 1994; 372:103-7];
Scid (scid) [Bosma GC et al. Nature 1983; 301:527-30; Mosier DE et al. Nature 1988; 335:256-9; and Greiner DL et al. Stem Cells 1998; 16:166-77];
NOD [Kikutani H et al. Adv Immunol 1992; 51:285-322; and Anderson MS et al. Ann Rev Immunol 2005; 23:447-85];
RAG1 and RAG2 (rag) [Mombaerts P et al. Cell 1992; 68:869-77; Shinkai U et al. Cell 1992; 68:855-67];
NOD-scid [Greiner DL et al. 1998; Shultz LD et al. J Immunol 1995; 154:180-91; Melkus MW et al. Nature Med 2006; 12:1316-22; and Denton PW et al. PLOS Med 2008; 4 (12): e357];
IL2rgnull [DiSanto JP et al. Proc Natl Acad Sci USA 1995; 92:377-81];
B2mnull [Christianson SW et al. J Immunol 1997; 158:3578-86];
NOD-scid IL2rynull [Shultz LD et al. Nat Rev Immunol 2007; 7:118-30; Ito M et al. Blood 2002; 100:3175-82; Ishikawa I et al. Blood 2005; 106:1565-73; and Macchiarini F et al. J Exp Med 2005; 202:1307-11];
NOD-scid B2mnull [Shultz et al. 2007; Shultz LD et al. Transplantation 2003; 76:1036-42; Islas-Ohlmayer MA et al. J Virol 2004; 78:13891-900; and Macchiarini et al. 2005];and
HLA transgenic mice [Grusby MJ et al. Proc Natl Acad Sci USA 1993; 90 (9): 3913-7;and Roy CJ et al. Infect Immun 2005; 73 (4): 2452-60]. See, e.g., Belizario JE The Open Immunology Journal, 2009; 2:79-85.

Provided herein, in some embodiments, are immunodeficient mouse models having the non-obese diabetic (NOD) mouse genotype. The NOD mouse (e.g., Jackson Labs Stock #001976, NOD-Shi^{LtJ}) is a polygenic mouse model of autoimmune (e.g., Type 1) diabetes, characterized by hyperglycemia and insulitis, a leukocytic infiltration of the pancreatic islet cells. The NOD mice are hypoinsulinemic and hyperglucagonemic, indicating a selective destruction of pancreatic islet beta cells. The major component of diabetes susceptibility in NOD mice is the unique MHC haplotype. NOD mice also exhibit multiple aberrant immunophenotypes including defective antigen presenting cell immunoregulatory functions, defects in the regulation of the T lymphocyte repertoire, defective NK cell function, defective cytokine production from macrophages (Fan et al., 2004) and impaired wound healing. They also lack hemolytic complement, C5. NOD mice also are severely hard-of-hearing. A variety of mutations causing immunodeficiencies, targeted mutations in cytokine genes, as well as transgenes affecting immune functions, have been backcrossed into the NOD inbred strain background.

In some aspects of the present disclosure, an immunodeficient mouse provided herein based on the NOD background may have a genotype selected from NOD-Cg.-Prkdc^{scid}IL2rg^{tm1wJl}/SzJ (NSG™), a NOD.Cg-Rag1^{tm1Mom} Il2rg^{tm1Wjl}/SzJ (NRG), and NOD.Cg-Prkdc^{scid}Il2rg^{tm1Sug}/ShiJic (NOG). Other immunodeficient mouse strains are contemplated herein.

In some embodiments, an immunodeficient mouse model has an NSG™ genotype. The NSG™ mouse (e.g., Jackson Labs Stock No.: #005557) is an immunodeficient mouse that lacks mature T cells, B cells, and NK cells, is deficient in multiple cytokine signaling pathways, and has many defects in innate immune immunity (see, e.g., Shultz, Ishikawa, & Greiner, 2007; Shultz et al., 2005; and Shultz et al., 1995, each of which is incorporated herein by reference). The NSG™ mouse, derived from the NOD mouse strain NOD/ShiLtJ (see, e.g., Makino et al., 1980, which is incorporated herein by reference), includes the Prkdc^{scid} mutation (also referred to as the “severe combined immunodeficiency” mutation or the “scid” mutation) and the Il2rg^{tm1Wjl} targeted mutation. The Prkdc^{scid} mutation is a loss-of-function (null) mutation in the mouse homolog of the human PRKDC gene—this mutation essentially eliminates adaptive immunity (see, e.g., (Blunt et al., 1995; Greiner, Hesselton, & Shultz, 1998), each of which is incorporated herein by reference). The Il2rg^{tm1Wjl} mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells (Cao et al., 1995; Greiner et al., 1998; and Shultz et al., 2005, each of which is incorporated herein by reference).

In some embodiments, an immunodeficient mouse model has an NRG genotype. The NRG mouse (e.g., Jackson Labs Stock #007799) is extremely immunodeficient. This mouse comprises two mutations on the NOD/ShiLtJ genetic background; a targeted knockout mutation in recombination activating gene 1 (Rag1) and a complete null allele of the IL2 receptor common gamma chain (IL2rg^{null}). The Rag1^{null} mutation renders the mice B and T cell deficient and the IL2rg^{null} mutation prevents cytokine signaling through multiple receptors, leading to a deficiency in functional NK cells. The extreme immunodeficiency of NRG allows the mice to be humanized by engraftment of human CD34⁺ hematopoietic stem cells (HSC) and patient derived xenografts (PDXs) at high efficiency. The immunodeficient NRG mice are more resistant to irradiation and genotoxic drugs than mice with a scid mutation in the DNA repair enzyme Prkdc.

In some embodiments, an immunodeficient mouse model is an NOG mouse. The NOG mouse (Ito M et al., Blood 2002) is an extremely severe combined immunodeficient (scid) mouse established by combining the NOD/scid mouse and the IL-2 receptor-γ chain knockout (IL2rγKO) mouse (Ohbo K. et al., Blood 1996). The NOG mouse lacks T and B cells, lacks natural killer (NK) cells, exhibits reduced dendritic cell function and reduced macrophage function, and lacks complement activity.

In some embodiments, an immunodeficient mouse model has an NCG genotype. The NCG mouse (e.g., Charles River Stock #572) was created by sequential CRISPR/Cas9 editing of the Prkdc and Il2rg loci in the NOD/Nju mouse, generating a mouse coisogenic to the NOD/Nju. The NOD/Nju carries a mutation in the Sirpa (SIRPα) gene that allows for engrafting of foreign hematopoietic stem cells. The Prkdc knockout generates a SCID-like phenotype lacking proper T-cell and B-cell formation. The knockout of the Il2rg gene further exacerbates the SCID-like phenotype while additionally resulting in a decrease of NK cell production.

Provided herein, in some embodiments, are immunodeficient mouse models that are deficient in MHC Class I, MHC Class II, or MHC Class I and MHC Class II. A mouse that is deficient in MHC Class I and/or MHC Class II does not express the same level of MHC Class I proteins (e.g., α-microglobulin and β2-microglobulin (B2M)) and/or MHC Class II proteins (e.g., α chain and β chain) or does not have the same level of MHC Class I and/or MHC Class II protein activity as a non-immunodeficient (e.g., MHC Class I/II wild-type) mouse. In some embodiments, the expression or activity of MHC Class I and/or MHC Class II proteins is reduced (e.g., by at least 30%, at least 40%, at least 50%, at least 60%, at least 70%, at least 80%, at least 90% or more), relative to a non-immunodeficient mouse.

Immunodeficient mice that are deficient in MHC Class I, MHC Class II, and MHC Class I and MHC Class II are described in International Publication No. WO 2018/209344, the contents of which are incorporated by reference herein.

The terms “MHC Class I” and “MHC I” are used interchangeably herein and refer to a complex formed by MHC I α protein and β2-microglobulin. MHC Class I α proteins includes an extracellular domain with the subdomains α1, α2, and α3, a transmembrane domain, and a cytoplasmic tail. The terms “H2-K”, “H2-D”, and “H2-L” refer to MHC Class I α protein subclasses, all of which are encoded on mouse chromosome 17. β2-microglobulin associates noncovalently with the α3 subdomain of MHC I α protein. The gene encoding mouse β2-microglobulin is encoded on mouse chromosome 2.

The terms “MHC Class II” and “MHC II” are used interchangeably to refer to a complex formed by two non-covalently associated proteins: an MHC II α protein and an MHC II β protein. The terms “H-2A” and “H-2E” (often abbreviated as I-A and I-E, respectively) refer to subclasses of MHC II. The MHC II α protein and MHC II β proteins span the plasma membrane and each contains an extracellular domain, a transmembrane domain, and a cytoplasmic domain. The extracellular portion of the MHC II α protein includes MHC II α1 and MHC II α2 domains, and the extracellular portion of the MHC II β protein includes MHC II β1 and MHC II β2 domains.

In some embodiments, an immunodeficient mouse deficient in MHC class I and MHC class II is a NOD.Cg-Prkdc^{scid} H2-K1^{tm1Bpe} H2-Ab1^{em1Mvw} H2-D1^{tm1Bpe Il}2rg^{tm1Wjl}/SzJ (abbreviated as NSG-(K^{b} D^{b})^{null} (IA^{null})). The NSG-(K^{b} D^{b})^{null} (IA^{null}) mouse lacks functional MHC I due to a homozygous null mutation of H2-K and H2-D MHC I α protein subclasses (abbreviated (K^{b} D^{b})^{null}). The NSG-(K^{b} D^{b})^{null} (IA^{null}) mouse lacks functional MHC II due to a homozygous null mutation of H-2A subclass of MHC II (abbreviated as IA^{null}).

In some embodiments, an immunodeficient mouse deficient in MHC class I and MHC class II is a NOD.Cg-Prkdc^{scid} H2-K1^{tm1Bpe} H2-Ab1^{em1Mvw} H2-D1^{tm1Bpe} Il2rg^{tm1Wjl} Tg(Ins2-HBEGF)6832Ugfm/Sz (abbreviated as NSG-B2M^{null} (IA IE)^{null}) mouse. The NSG-B2M^{null} (IA IE)^{null} mouse lacks functional MHC I due to a homozygous null mutation of β2 microglobulin (abbreviated B2M^{null}). The NSG-B2M^{null} (IA IE)^{null} mouse lacks functional MHC II due to a homozygous null mutation of H-2A and H-2E subclasses of MHC II (abbreviated as (IA IE)^{null}).

In some embodiments, an immunodeficient mouse deficient in MHC class I and MHC class II is a NOD.Cg-Prkdc^{scid} H2-K1^{tm1Bpe} H2-Ab1^{em1Mvw} H2-D1^{tm1Bpe} Il2rg^{tm1Wjl} Tg(Ins2-HBEGF)6832Ugfm/Sz transgenic mouse, abbreviated as NSG-RIP-DTR (K^{b} D^{b})^{null} (IA^{null}), which expresses the diphtheria toxin receptor under the control of the rat insulin promoter on an NSG™ background. Injection of diphtheria toxin (DT) into mice expressing the diphtheria toxin receptor under the control of the rat insulin promoter leads to mouse pancreatic beta cell death and hyperglycemia. The NSG-RIP-DTR (K^{b} D^{b})^{null} (IA^{null}) strain permits the complete and specific ablation of mouse pancreatic beta cells, avoiding the broadly toxic effects of diabetogenic drugs such as streptozotocin.

### Humanized Mouse Models

Provided herein, in some embodiments, are humanized immunodeficient mouse models and methods of producing the models. Immunodeficient mice engrafted with functional human cells and/or tissues are referred to as “humanized mice.” As used herein, the terms “humanized mouse”, “humanized immune deficient mouse”, “humanized immunodeficient mouse”, and the plural versions thereof are used interchangeably to refer to an immunodeficient mouse humanized by engraftment with functional human cells and/or tissues. For example, mouse models may be engrafted with human hematopoietic stem cells (HSCs) and/or human peripheral blood mononuclear cells (PMBCs). In some embodiments, mouse models are engrafted with human tissues such as islets, liver, skin, and/or solid or hematologic cancers. In other embodiments, mouse models may be genetically modified such that endogenous mouse genes are converted to human homologs (see, e.g., Pearson, et al., Curr Protoc Immunol., 2008,Chapter: Unit-15.21).

Humanized mice are generated by starting with an immunodeficient mouse and, if necessary, depleting and/or suppressing any remaining murine immune cells (e.g., chemically or with radiation). That is, successful survival of the human immune system in the immunodeficient mice may require suppression of the mouse's immune system to prevent GVHD (graft-versus-host disease) rejections. After the immunodeficient mouse's immune system has been sufficiently suppressed, the mouse is engrafted with human cells (e.g., HSCs and/or PBMCs). As used herein, “engraft” refers to the process of the human cells migrating to, and incorporating into, an existing tissue of interest in vivo. With respect to the humanized immunodeficient mouse, the engrafted human cells provide functional mature human cells (e.g., immune cells). The model has a specific time window of about 4-5 weeks after engraftment before GVHD sets in. To increase the longevity of the model, double-knockout mice lacking functional MHC I and MHC II, as described above, may be used.

The engrafted human cells (e.g., HSCs or PMBCs) for humanization, in some embodiments, are human leukocyte-antigen (HLA)-matched to the human cancer cells of the mouse models. HLA-matched refers to cells that express the same major histocompatibility complex (MHC) genes. Engrafting mice with HLA-matched human xenografts and human immune cells, for example, reduces or prevents immunogenicity of the human immune cells. In some embodiments, a humanized mouse provided in the present disclosure is engrafted with human PMBCs or human HSCs that are HLA-matched to a PDX or human cancer cell line.

### Irradiation

As described above, in some embodiments, immunodeficient mice are irradiated prior to engraftment with human cells, such as human HSCs and/or PMBCs. It is thought that irradiation of an immunodeficient mouse destroys mouse immune cells in peripheral blood, spleen, and bone marrow, which facilitates engraftment of human cells, such as human HSCs and/or PMBCs (e.g., by increasing human cell survival factors), as well as expansion of other immune cells. Irradiation also shortens the time it takes to accumulate the required number of human immune cells to “humanize” the mouse models.

For immunodeficient mice (e.g., NSG™ mice), this preparation is commonly accomplished through whole-body gamma irradiation. Irradiators may vary in size depending on their intended use. Animals are generally irradiated for short periods of time (less than 15 min). The amount of time spent inside the irradiator varies depending on the radioisotope decay charts, amount of irradiation needed, and source of ionizing energy (that is, X-rays versus gamma rays, for which a cesium or cobalt source is needed).

A myeloablative irradiation dose is usually 700 to 1300 cGy, though in some embodiments, lower doses such as 1-100 cGy (e.g., about 2, 5, or 10 cGy), or 300-700 cGy may be used.

As an example, the mouse may be irradiated with 100 cGy X-ray (or 75 cGy-125 cGy X-ray). In some embodiments, the dose is about 1, 2, 3, 4, 5, 10, 20, 100, 200, 300, 400, 500, 600, 700, 800, 900, 1000, 1100, 1200, or 1300 cGy, or between any of the two recited doses herein, such as 100-300 cGy, 200-500 cGy, 600-1000 cGy, or 700-1300 cGy. In some embodiments, the immunodeficient mouse is irradiated about 15 minutes, 30 minutes, 45 minutes, 1 hour, or more before engraftment with human HSCs and/or PMBCs. In some embodiments, the immunodeficient mouse is engrafted with human HSCs and/or PMBCs 1, 2, 3, 4, 5, 6, 7, 8, 9, 10, 11, 12, 13, 14, 15, 16, 17, or 18 days after irradiation.

### Engraftment

As described above, in some embodiments, the irradiated immunodeficient mice are engrafted with HSCs and/or PBMCs, humanizing the mice. Engraftment refers to the process of the human cells migrating to, and incorporating into, an existing tissue of interest in vivo. The PBMCs may be engrafted after irradiation and before engraftment of human cancer cells, after irradiation and concurrently with engraftment of human cancer cells, or after irradiation and after engraftment of human cancer cells.

Peripheral blood mononuclear cells (PBMCs) are peripheral blood cells having a round nucleus. These mononuclear blood cells recirculate between tissues and blood and are a critical component in the immune system to fight infection and adapt to intruders. There are two main types of mononuclear cells, lymphocytes and monocytes. The lymphocyte population of PBMCs typically includes T cells, B cells and NK cells.

PBMCs may be isolated from whole blood samples, for example (e.g., Ficoll gradient). In some embodiments, PBMCs from a subject (e.g., a human subject) with a current or previous diagnosis of a pathogen or pathogenic disease may be used.

Hematopoietic stem cells (HSCs) are the stem cells that give rise to other blood cells during a process referred to as hematopoiesis. Hematopoietic stem cells give rise to different types of blood cells, in lines called myeloid and lymphoid. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, natural killer cells, and innate lymphoid cells.

Methods of engrafting immunodeficient mice with HSCs and/or PBMCs to yield a humanized mouse model include but are not limited to intraperitoneal or intravenous injection (Shultz et al., J Immunol, 2015, 174:6477-6489; Pearson et al., Curr Protoc Immunol. 2008; 15-21; Kim et al., AIDS Res Hum Retrovirus, 2016, 32 (2): 194-2020; Yaguchi et al., Cell & Mol Immunol, 2018, 15:953-962). In some embodiments, the mouse is engrafted with 1.0×10⁶-3.0×10⁷ HSCs and/or PBMCs.

For example, the mouse may be engrafted with 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.5×10⁶, 3.0×10⁶ or more HSCs and/or PBMCs. In some embodiments, the mouse is engrafted with 1.0-1.1×10⁶, 1.0-1.2×10⁶, 1.0-1.3×10⁶, 1.0-1.4×10⁶, 1.0-1.5×10⁶, 1.0-1.6×10⁶, 1.0-1.7×10⁶, 1.0-1.8×10⁶, 1.0-1.9×10⁶, 1.0-2.0×10⁶, 1.0-2.25×10⁶, 1.0-2.5×10⁶, 1.0-2.75×10⁶, 1.0-3.0×10⁶, 1.1-1.2×10⁶, 1.1-1.3×10⁶, 1.1-1.4×10⁶, 1.1-1.5×10⁶, 1.1-1.6×10⁶, 1.1-1.7×10⁶, 1.1-1.8×10⁶, 1.1-1.9×10⁶, 1.1-2.0×10⁶, 1.1-2.25×10⁶, 1.1-2.5×10⁶, 1.1-2.75×10⁶, 1.1-3.0×10⁶, 1.2-1.3×10⁶, 1.2-1.4×10⁶, 1.2-1.5×10⁶, 1.2-1.6×10⁶, 1.2-1.7×10⁶, 1.2-1.8×10⁶, 1.2-1.9×10⁶, 1.2-2.0×10⁶, 1.2-2.25×10⁶, 1.2-2.5×10⁶, 1.2-2.75×10⁶, 1.2-3.0×10⁶, 1.3-1.4×10⁶, 1.3-1.5×10⁶, 1.3-1.6×10⁶, 1.3-1.7×10⁶, 1.3-1.8×10⁶, 1.3-1.9×10⁶, 1.3-2.0×10⁶, 1.3-2.25×10⁶, 1.3-2.5×10⁶, 1.3-2.75×10⁶, 1.3-3.0×10⁶, 1.4-1.5×10⁶, 1.4-1.6×10⁶, 1.4-1.7×10⁶, 1.4-1.8×10⁶, 1.4-1.9×10⁶, 1.4-2.0×10⁶, 1.4-2.25×10⁶, 1.4-2.5×10⁶, 1.4-2.75×10⁶, 1.4-3.0×10⁶, 1.5-1.6×10⁶, 1.5-1.7×10⁶, 1.5-1.8×10⁶, 1.5-1.9×10⁶, 1.5-2.0×10⁶, 1.5-2.25×10⁶, 1.5-2.5×10⁶, 1.5-2.75×10⁶, 1.5-3.0×10⁶, 1.6-1.7×10⁶, 1.6-1.8×10⁶, 1.6-1.9×10⁶, 1.6-2.0×10⁶, 1.6-2.25×10⁶, 1.6-2.5×10⁶, 1.6-2.75×10⁶, 1.6-3.0×10⁶, 1.7-1.8×10⁶, 1.7-1.9×10⁶, 1.7-2.0×10⁶, 1.7-2.25×10⁶, 1.7-2.5×10⁶, 1.7-2.75×10⁶, 1.7-3.0×10⁶, 1.8-1.9×10⁶, 1.8-2.0×10⁶, 1.8-2.25×10⁶, 1.8-2.5×10⁶, 1.8-2.75×10⁶, 1.8-3.0×10⁶, 1.9-2.0×10⁶, 1.9-2.25×10⁶, 1.9-2.5×10⁶, 1.9-2.75×10⁶, 1.9-3.0×10⁶, 2.0-2.25×10⁶, 2.0-2.5×10⁶, 2.0-2.75×10⁶, 2.0-3.0×10⁶, 2.25-2.5×10⁶, 2.25-2.75×10⁶, 2.25-3.0×10⁶, 2.5-2.75×10⁶, 2.5-3.0×10⁶, or 2.75-3.0×10⁶ HSCs and/or PBMCs.

In some embodiments, the mouse may be engrafted with 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.5×10⁷, 3.0×10⁷ or more HSCs and/or PBMCs. In some embodiments, the mouse is engrafted with 1.0-1.1×10⁷, 1.0-1.2×10⁷, 1.0-1.3×10⁷, 1.0-1.4×10⁷, 1.0-1.5×10⁷, 1.0-1.6×10⁷, 1.0-1.7×10⁷, 1.0-1.8×10⁷, 1.0-1.9×10⁷, 1.0-2.0×10⁷, 1.0-2.25×10⁷, 1.0-2.5×10⁷, 1.0-2.75×10⁷, 1.0-3.0×10⁷, 1.1-1.2×10⁷, 1.1-1.3×10⁷, 1.1-1.4×10⁷, 1.1-1.5×10⁷, 1.1-1.6×10⁷, 1.1-1.7×10⁷, 1.1-1.8×10⁷, 1.1-1.9×10⁷, 1.1-2.0×10⁷, 1.1-2.25×10⁷, 1.1-2.5×10⁷, 1.1-2.75×10⁷, 1.1-3.0×10⁷, 1.2-1.3×10⁷, 1.2-1.4×10⁷, 1.2-1.5×10⁷, 1.2-1.6×10⁷, 1.2-1.7×10⁷, 1.2-1.8×10⁷, 1.2-1.9×10⁷, 1.2-2.0×10⁷, 1.2-2.25×10⁷, 1.2-2.5×10⁷, 1.2-2.75×10⁷, 1.2-3.0×10⁷, 1.3-1.4×10⁷, 1.3-1.5×10⁷, 1.3-1.6×10⁷, 1.3-1.7×10⁷, 1.3-1.8×10⁷, 1.3-1.9×10⁷, 1.3-2.0×10⁷, 1.3-2.25×10⁷, 1.3-2.5×10⁷, 1.3-2.75×10⁷, 1.3-3.0×10⁷, 1.4-1.5×10⁷, 1.4-1.6×10⁷, 1.4-1.7×10⁷, 1.4-1.8×10⁷, 1.4-1.9×10⁷, 1.4-2.0×10⁷, 1.4-2.25×10⁷, 1.4-2.5×10⁷, 1.4-2.75×10⁷, 1.4-3.0×10⁷, 1.5-1.6×10⁷, 1.5-1.7×10⁷, 1.5-1.8×10⁷, 1.5-1.9×10⁷, 1.5-2.0×10⁷, 1.5-2.25×10⁷, 1.5-2.5×10⁷, 1.5-2.75×10⁷, 1.5-3.0×10⁷, 1.6-1.7×10⁷, 1.6-1.8×10⁷, 1.6-1.9×10⁷, 1.6-2.0×10⁷, 1.6-2.25×10⁷, 1.6-2.5×10⁷, 1.6-2.75×10⁷, 1.6-3.0×10⁷, 1.7-1.8×10⁷, 1.7-1.9×10⁷, 1.7-2.0×10⁷, 1.7-2.25×10⁷, 1.7-2.5×10⁷, 1.7-2.75×10⁷, 1.7-3.0×10⁷, 1.8-1.9×10⁷, 1.8-2.0×10⁷, 1.8-2.25×10⁷, 1.8-2.5×10⁷, 1.8-2.75×10⁷, 1.8-3.0×10⁷, 1.9-2.0×10⁷, 1.9-2.25×10⁷, 1.9-2.5×10⁷, 1.9-2.75×10⁷, 1.9-3.0×10⁷, 2.0-2.25×10⁷, 2.0-2.5×10⁷, 2.0-2.75×10⁷, 2.0-3.0×10⁷, 2.25-2.5×10⁷, 2.25-2.75×10⁷, 2.25-3.0×10⁷, 2.5-2.75×10⁷, 2.5-3.0×10⁷, or 2.75-3.0×10⁷ HSCs and/or PBMCs. In some embodiments, the mouse is engrafted with 2×10⁷ HSCs and/or PBMCs. According to some embodiments, the mouse is engrafted with 4.5-5.5×10⁷ (4.5-5.0×10⁷, 5.0-5.5×10⁷) HSCs and/or PBMCs.

### Nucleic Acids: Engineering and Delivery

The nucleic acids provided herein, in some embodiments, are engineered. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D.G. et al. Nature Methods, 343-345, 2009; and Gibson, D.G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences).

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene).

A promoter is a nucleotide sequence to which RNA polymerase binds to initial transcription (e.g., ATG). Promoters are typically located directly upstream from (at the 5′ end of) a transcription initiation site. In some embodiments, a promoter is an endogenous promoter. An endogenous promoter is a promoter that naturally occurs in that host animal.

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

An exon is a region of a gene that codes for amino acids. An intron (and other non-coding DNA) is a region of a gene that does not code for amino acids.

A nucleotide sequence encoding a product (e.g., protein), in some embodiments, has a length of 200 base pairs (bp) to 100 kilobases (kb). The nucleotide sequence, in some embodiments, has a length of at least 10 kb. For example, the nucleotide sequence may have a length of at least 15 kb, at least 20 kb, at least 25 kb, at least 30 kb, or at least 35 kb. In some embodiments, the nucleotide sequence has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb.

Any one of the nucleic acids provided herein may have a length of 200 bp to 500 kb, 200 bp to 250 kb, or 200 bp to 100 kb. A nucleic acid, in some embodiments, has a length of at least 10 kb. For example, a nucleic acid may have a length of at least 15 kb, at least 20 kb, at least 25 kb, at least 30 kb, at least 35 kb, at least 50 kb, at least 100 kb, at least 200 kb, at least 300 kb, at least 400 kb, or at least 500 kb. In some embodiments, a nucleic acid has a length of 10 to 500 kb, 20 to 400 kb, 10 to 300 kb, 10 to 200 kb, or 10 to 100 kb. In some embodiments, a nucleic acid has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A nucleic acid may be circular or linear.

The nucleic acids described herein, in some embodiments, include a modification. A modification, with respect to a nucleic acid, is any manipulation of the nucleic acid, relative to the corresponding wild-type nucleic acid (e.g., the naturally-occurring nucleic acid). A genomic modification is thus any manipulation of a nucleic acid in a genome (e.g., in a coding region, non-coding region, and/or regulatory region), relative to the corresponding wild-type nucleic acid (e.g., the naturally-occurring (unmodified) nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g., protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs).

A nucleic acid, such as an allele or alleles of a gene, may be modified such that it does not produce a detectable level of a functional gene product (e.g., a functional protein). Thus, an inactivated allele is an allele that does not produce a detectable level of a functional gene product (e.g., a functional protein). A detectable level of a protein is any level of protein detected using a standard protein detection assay, such as flow cytometry and/or an ELISA. In some embodiments, an inactivated allele is not transcribed. In some embodiments, an inactivated allele does not encode a functional protein.

Vectors used for delivery of a nucleic acid include minicircles, plasmids, bacterial artificial chromosomes (BACs), and yeast artificial chromosomes. It should be understood, however, that a vector may not be needed. For example, a circularized or linearized nucleic acid may be delivered to an embryo without its vector backbone. Vector backbones are small (˜4 kb), while donor DNA to be circularized can range from >100 bp to 50 kb, for example.

Methods for delivering nucleic acids to mouse embryos for the production of transgenic mice include, but are not limited to, electroporation (see, e.g., Wang W et al. J Genet Genomics 2016;43 (5): 319-27; WO 2016/054032; and WO 2017/124086, each of which is incorporated herein by reference), DNA microinjection (see, e.g., Gordon and Ruddle, Science 1981:214: 1244-124, incorporated herein by reference), embryonic stem cell-mediated gene transfer (see, e.g., Gossler et al., Proc. Natl. Acad. Sci. 1986; 83:9065-9069, incorporated herein by reference), and retrovirus-mediated gene transfer (see, e.g., Jaenisch, Proc. Natl. Acad. Sci. 1976; 73:1260-1264, incorporated herein by reference), any of which may be used as provided herein.

### Genomic Editing Methods

Engineered nucleic acids, such as guide RNAs, donor polynucleotides, and other nucleic acid coding sequences, for example, may be introduced to a genome of an embryo using any suitable method. The present application contemplates the use of a variety of gene editing technologies, for example, to introduce nucleic acids into the genome of an embryo to produce a transgenic rodent. Non-limiting examples include programmable nuclease-based systems, such as clustered regularly interspaced short palindromic repeat (CRISPR) systems, zinc-finger nucleases (ZFNs), and transcription activator-like effector nucleases (TALENs). See, e.g., Carroll D Genetics. 2011; 188 (4): 773-782; Joung JK et al. Nat Rev Mol Cell Biol. 2013; 14 (1): 49-55; and Gaj T et al. Trends Biotechnol. 2013 July; 31 (7): 397-405, each of which is incorporated by reference herein.

In some embodiments, a CRISPR system is used to edit the genome of mouse embryos provided herein. See, e.g., Harms DW et al., Curr Protoc Hum Genet. 2014; 83:15.7.1-15.7.27; and Inui M et al., Sci Rep. 2014; 4:5396, each of which are incorporated by reference herein). For example, Cas9 mRNA or protein, one or multiple guide RNAs (gRNAs), and/or a donor nucleic acid can be delivered, e.g., injected or electroporated, directly into mouse embryos at the one-cell (zygote) stage or a later stage to facilitate homology directed repair (HDR), for example, to introduce an engineered nucleic acid (e.g., donor nucleic acid) into the genome.

The CRISPR/Cas system is a naturally occurring defense mechanism in prokaryotes that has been repurposed as an RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the Cas9 endonuclease is from Streptococcus pyogenes (NGG PAM) or Staphylococcus aureus (NNGRRT or NNGRR(N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitidis (NM) Cas9 (NNNNGATT PAM); Streptococcus thermophilus (ST) Cas9 (NNAGAAW PAM); and Treponema denticola (TD) Cas9 (NAAAAC). In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence. See, e.g., Jinek et al., Science, 2012; 337:816-821 and Deltcheva et al., Nature, 2100; 471:602-607, each of which is incorporated by reference herein.

In some embodiments, the RNA-guided nuclease and the gRNA are complexed to form a ribonucleoprotein (RNP), prior to delivery to an embryo.

The concentration of RNA-guided nuclease or nucleic acid encoding the RNA-guided nuclease may vary. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl. In some embodiments, the concentration is 100 ng/μl to 500 ng/μl, or 200 ng/μl to 500 ng/μl.

The concentration of gRNA may also vary. In some embodiments, the concentration is 200 ng/μl to 2000 ng/μl. For example, the concentration may be 200, 300, 400, 500, 600, 700, 800, 900, 1000, 1100, 1200, 1300, 1400, 1500, 1600, 1700, 1700, 1900, or 2000 ng/μl. In some embodiments, the concentration is 500 ng/μl to 1000 ng/μl. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl.

In some embodiments, the ratio of concentration of RNA-guided nuclease or nucleic acid encoding the RNA-guided nuclease to the concentration of gRNA is 2:1. In other embodiments, the ratio of concentration of RNA-guided nuclease or nucleic acid encoding the RNA-guided nuclease to the concentration of gRNA is 1:1.

A donor nucleic acid typically includes a sequence of interest flanked by homology arms. Homology arms are regions of the ssDNA that are homologous to regions of genomic DNA located in a genomic locus. One homology arm is located to the left (5′) of a genomic region of interest (into which a sequence of interest is introduced) (the left homology arm) and another homology arm is located to the right (3′) of the genomic region of interest (the right homology arm). These homology arms enable homologous recombination between the ssDNA donor and the genomic locus, resulting in insertion of the sequence of interest into the genomic locus of interest (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded. In some embodiments, the donor DNA is modified, e.g., via phosphorothioation. Other modifications may be made.

### Methods of Use

The mouse models of the present disclosure may be used, in some embodiments, to assess the half-life of a therapeutic antibody or other therapeutic protein. Thus, in some embodiments, a method comprising administering to the mouse model a therapeutic antibody (or other therapeutic protein) and assaying half-life of the antibody in a biological sample from the mouse.

The elimination half-life (e.g., serum half-life) of a drug is a pharmacokinetic parameter that is defined as the time it takes for the concentration of the drug in the plasma or the total amount in the body to be reduced by 50%. Thus, after one half-life, the concentration of the drug in the body will be half of the starting dose. For example, if a 100 mg dose of a drug with a half-life of 15 minute is administered intravenously, 15 minutes after administration, 50 mg of the drug remains in the body. In general, the effect of the drug is considered to have a negligible therapeutic effect after 4 half-lives, that is, when only 6.25% of the original dose remains in the body.

The mouse models of the present disclosure may also or alternatively be used, in some embodiments, to identify candidate therapeutic antibodies with desirable therapeutic properties. Non-limiting examples of desirable therapeutic properties include long half-life (≥7 days, see, e.g., Ryman and Meibohm, Pharmacokinetics of monoclonal antibodies, CPT Pharmacometrics Syst Pharmacol, 2017, 6 (9): 576-588), low immunogenicity, specific binding to target molecules, solubility in the body, and conformational stability.

Non-limiting examples of routes of administration include intravenous, intraperitoneal, subcutaneous, and intramuscular. Other routes of administration are encompassed by the present disclosure.

A biological sample, in some embodiments, is a blood sample, such as a serum or plasma sample. Other biological samples are encompassed by the present disclosure.

Non-limiting examples of methods for assaying half-life of a therapeutic protein include enzyme linked immunosorbent assay (ELISA), surface plasmon resonance (SPR), radioimmunoassay, chemiluminescent immunoassay, and immunoradiometric assay. Other methods of measuring protein levels in a biological sample are encompassed by the present disclosure.

The therapeutic protein, in some embodiments, is an antibody, such as a monoclonal antibody. In some embodiments, an antibody is a human antibody. In some embodiments, an antibody is a humanized antibody. Humanized antibodies combine a human antibody with a small part of a mouse or rat monoclonal antibody. In some embodiments, an antibody is a chimeric antibody. Chimeric antibodies are structural chimeras made by fusing variable regions from one species (e.g., a mouse) with the constant regions from another species (e.g., a human).

An IgG antibody is one example of an antibody that may be assessed using the mouse models provided herein. For example, the antibody may be an IgG1, IgG2, IgG3, or lgG4antibody. In some embodiments, the therapeutic antibody is a monoclonal antibody, for example, a monoclonal IgG antibody.

In some embodiments, the mouse models provided herein are used to assess PK behavior of preclinical antibody and/or human Fc fusion candidates. Assessment of other preclinical protein candidates is also contemplated herein.

Non-limiting examples of antibodies that may be assessed using the models of the present disclosure include abagovomab, abciximab, abituzumab, abrezekimab, abrilumab, actoxumab, adalimumab, adecatumumab, aducanumab, afasevikumab, afelimomab, alacizumab pegol, alemtuzumab, alirocumab, altumomab pentetate, amatuximab, amivantamab, anatumomab mafenatox, andecaliximab, anetumab ravtansine, anifrolumab, anrukinzumab, apolizumab, aprutumab ixadotin, arcitumomab, ascrinvacumab, aselizumab, atezolizumab, atidortoxumab, atinumab, atoltivimab, atoltivimab/maftivimab/odesivimab, atorolimumab, avelumab, azintuxizumab vedotin, bapincuzumab, basiliximab, bavituximab, bectumomab, begelomab, belantamab mafodotin, belimumab, bemarituzumab, benralizumab, berlimatoxumab, bermekimab, bersanlimab, bertilimumab, besilesomab, bevacizumab, bezlotoxumab, biciromab, bimagrumab, bimekizumab, birtamimab, bivatuzumab, bleselumab, blinatumomab, blontuvetmab, blosozumab, bococizumab, brazikumab, brentuximab vedotin, briakinumab, brodalumab, brolucizumab, brontictuzumab, burosumab, cabiralizumab, camidanlumab tesirine, camrelizumab, canakinumab, cantuzumab mertansine, cantuzumab ravtansine, caplacizumab, capromab, carlumab, carotuximab, catumaxomab, cedelizumab, cemiplimab, cergutuzumab amunaleukin, certolizumab pegol, cetrelimab, cetuximab, cibisatamab, cirmtuzumab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, crenezumab, crizanlizumab, crotedumab, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, dezamizumab, dinutuximab, dinutuximab beta, diridavumab, domagrozumab, dorlimomab aritox, dostarlimab, drozitumab, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, enapotamab vedotin, enavatuzumab, enfortumab vedotin, enlimomab pegol, enoblituzumab, enokizumab, enoticumab, ensituximab, epitumomab cituxctan, epratuzumab, eptinezumab, erenumab, erlizumab, ertumaxomab, etaracizumab, etigilimab, etrolizumab, evinacumab, evolocumab, exbivirumab, fanolesomab, faralimomab, faricimab, farletuzumab, fasinumab, fbta, felvizumab, fezakinumab, fibatuzumab, ficlatuzumab, figitumumab, firivumab, flanvotumab, fletikumab, flotetuzumab, fontolizumab, foralumab, foravirumab, fremanczumab, fresolimumab, frovocimab, frunevetmab, fulranumab, futuximab, galcanczumab, galiximab, gancotamab, ganitumab, gantenerumab, gatipotuzumab, gavilimomab, gedivumab, gemtuzumab ozogamicin, gevokizumab, gilvetmab, gimsilumab, girentuximab, glembatumumab vedotin, golimumab, gomiliximab, gosuranemab, guselkumab, ianalumab, ibalizumab, ibi, ibritumomab tiuxetan, icrucumab, idarucizumab, ifabotuzumab, igovomab, iladatuzumab vedotin, imab, imalumab, imaprelimab, imciromab, imgatuzumab, inclacumab, indatuximab ravtansine, indusatumab vedotin, inebilizumab, infliximab, intetumumab, inolimomab, inotuzumab ozogamicin, ipilimumab, iratumumab, isatuximab, iscalimab, istiratumab, itolizumab, ixckizumab, keliximab, labctuzumab, lacnotuzumab, ladiratuzumab vedotin, lampalizumab, lanadelumab, landogrozumab, laprituximab emtansine, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab vedotin, ligelizumab, loncastuximab tesirine, losatuxizumab vedotin, lilotomab satetraxctan, lintuzumab, lirilumab, lodelcizumab, lokivetmab, lorvotuzumab mertansine, lucatumumab, lulizumab pegol, lumiliximab, lumretuzumab, lupartumab, lupartumab amadotin, lutikizumab, maftivimab, mapatumumab, margetuximab, marstacimab, maslimomab, mavrilimumab, matuzumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxctumomab pasudotox, muromonab-cd, nacolomab tafenatox, namilumab, naptumomab estafenatox, naratuximab emtansinc, narnatumab, natalizumab, navicixizumab, navivumab, naxitamab, nebacumab, necitumumab, nemolizumab, neod, nerelimomab, nesvacumab, netakimab, nimotuzumab, nirsevimab, nivolumab, nofetumomab merpentan, obiltoxaximab, obinutuzumab, ocaratuzumab, ocrelizumab, odesivimab, odulimomab, ofatumumab, olaratumab, oleclumab, olendalizumab, olokizumab, omalizumab, omburtamab, oms, onartuzumab, ontuxizumab, onvatilimab, opicinumab, oportuzumab monatox, oregovomab, orticumab, otelixizumab, otilimab, otlertuzumab, oxclumab, ozanczumab, ozoralizumab, pagibaximab, palivizumab, pamrevlumab, panitumumab, pankomab, panobacumab, parsatuzumab, pascolizumab, pasotuxizumab, pateclizumab, patritumab, pdr, pembrolizumab, pemtumomab, perakizumab, pertuzumab, pexelizumab, pidilizumab, pinatuzumab vedotin, pintumomab, placulumab, prezalumab, plozalizumab, pogalizumab, polatuzumab vedotin, ponczumab, porgaviximab, prasinczumab, prezalizumab, priliximab, pritoxaximab, pritumumab, quilizumab, racotumomab, radretumab, rafivirumab, ralpancizumab, ramucirumab, ranevetmab, ranibizumab, raxibacumab, ravagalimab, ravulizumab, refanczumab, regavirumab, relatlimab, remtolumab, reslizumab, rilotumumab, rinucumab, risankizumab, rituximab, rivabazumab pegol, robatumumab, rmab, roledumab, romilkimab, romosozumab, rontalizumab, rosmantuzumab, rovalpituzumab tesirine, rovelizumab, rozanolixizumab, ruplizumab, sacituzumab govitecan, samalizumab, samrotamab vedotin, sarilumab, satralizumab, satumomab pendetide, secukinumab, selicrelumab, scribantumab, setoxaximab, setrusumab, sevirumab, sibrotuzumab, sifalimumab, siltuximab, simtuzumab, siplizumab, sirtratumab vedotin, sirukumab, sofituzumab vedotin, solanczumab, solitomab, sonepcizumab, sontuzumab, spartalizumab, stamulumab, sulesomab, suptavumab, sutimlimab, suvizumab, suvratoxumab, tabalumab, tacatuzumab tetraxetan, tadocizumab, tafasitamab, talacotuzumab, talizumab, talquetamab, tamtuvetmab, tanczumab, taplitumomab paptox, tarextumab, tavolimab, teclistamab, tefibazumab, telimomab aritox, telisotuzumab, telisotuzumab vedotin, tenatumomab, teneliximab, teplizumab, tepoditamab, teprotumumab, tesidolumab, tetulomab, tezepelumab, tibulizumab, tildrakizumab, tigatuzumab, timigutuzumab, timolumab, tiragolumab, tiragotumab, tislelizumab, tisotumab vedotin, tocilizumab, tomuzotuximab, toralizumab, tosatoxumab, tositumomab, tovetumab, tralokinumab, trastuzumab, trastuzumab duocarmazine, trastuzumab emtansine, tregalizumab, tremelimumab, trevogrumab, tucotuzumab celmolcukin, tuvirumab, ublituximab, ulocuplumab, urelumab, urtoxazumab, ustekinumab, utomilumab, vadastuximab talirine, vanalimab, vandortuzumab vedotin, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab mafodotin, votumumab, vunakizumab, xentuzumab, xmab-, zalutumumab, zanolimumab, zatuximab, zenocutuzumab, ziralimumab, zolbetuximab, and zolimomab aritox.

### EXAMPLES

### Example 1. Production of Mice Expressing Chimeric Mouse/Human IgG1 and Human FcRn

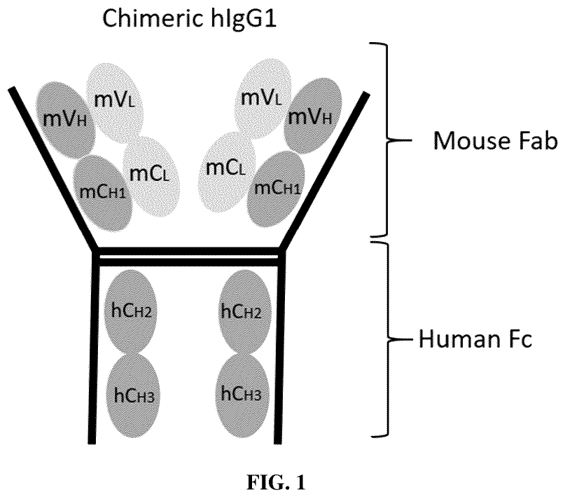

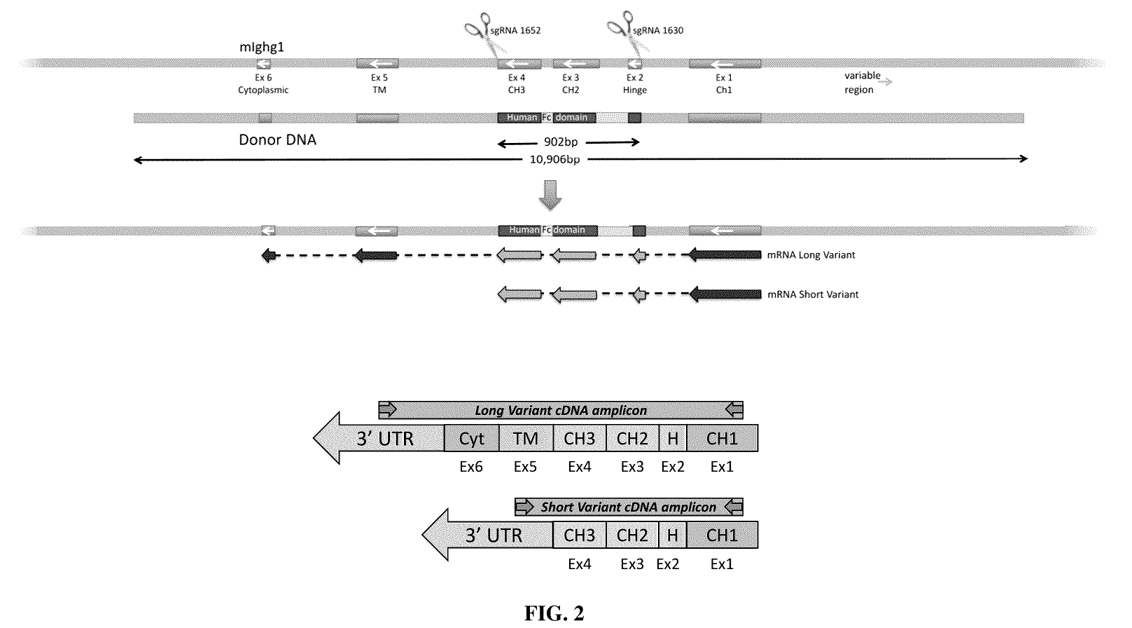

To produce mice expressing a chimeric mouse/human IgG1 and expressing human neonatal Fc receptor (FcRn), encoded by Fegrt, Fc receptor, IgG, alpha chain transporter, the B6.Cg-Fcgrt^{tm1Dcr}Tg(FCGRT)32Dcr/DcrJ (also referred to as Tg32) mouse strain as modified using CRISPR/Cas9 gene editing technology. Single guide RNAs (gRNAs) were designed to bind to the genomic region flanking the mouse immunoglobulin 1 (mIgG1) Fc and hinge locus. The donor DNA, carrying a human hinge and Fc sequence, included 2-3 kilobase (kb) homology arms flanking the mouse hinge and Fc sequence. Cas9 mRNA, the gRNAs (SEQ ID NO: 4 and SEQ ID NO: 5), and the donor DNA were injected into Tg32 mouse embryos followed by CRISPR-mediated homologous recombination to produce chimeric IgG1 (FIGS. 1 and 2).

Eighty-seven (87) live born mice were analyzed by polymerase chain reaction (PCR) for correct integration of the donor DNA (with human IgG1 Fc and hinge sequence). Three founder animals were identified, of which two transmitted the humanized Fc and hinge domain (B6.Cg-Fcgrt^{tm1Dcr} Ighg1^{em2(IGHG1)Mvw}Tg(FCGRT)32Dcr/Mvw; also referred to as Tg32-hFc, JAX Stock #029686), one of which, referred to herein asTg32-hFc, was used for further studies.

### Example 2. Modeling Human FcRn-Mediated Antibody Recycling

To assess whether the chimeric IgG1 cDNA was being synthesized in the Tg32-hFc mouse model provided herein, spleens were taken from mice of the Tg32-hFc strain and from mice of the Tg32 strain and subjected to RT-PCR. Chimeric IgG1 cDNA was detected in the Tg32-hFc mice, and endogenous mouse IgG1 cDNA was detected in the Tg32 mice. All results were verified by sequencing (data not shown).

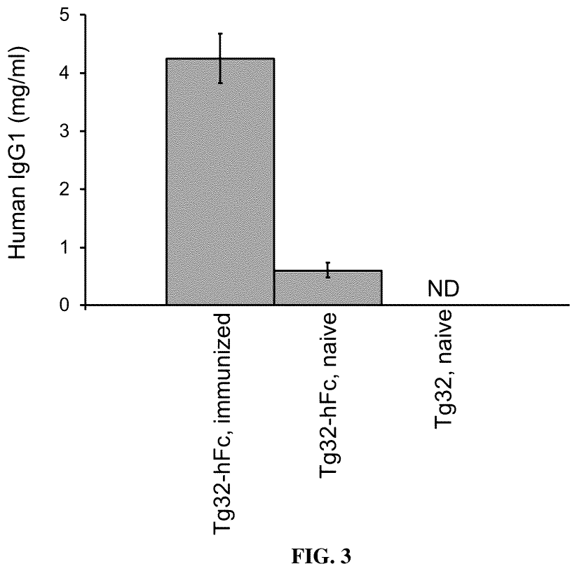

Mice of both the Tg32-hFc strain and the Tg32 strain express human FcRn at physiologically-relevant human levels from the endogenous human FCGRT promoter. For mice of the Tg32-hFc strain to mimic competition for human FcRn-mediated IgG1 recycling, serum chimeric IgG1 levels should also be physiologically relevant for humans. Serum chimeric IgG1 levels were measured in Tg32-hFc mice either immunized or non-immunized with 2,4-Dinitrophenyl hapten conjugated to keyhole limpet hemocyanin (DNP-KLH). Surprisingly, the serum IgG1 level was ˜4.8 mg/mL in non-immunized mice and rose to ˜29 mg/mL following immunization (FIG. 3). Serum IgG1 levels in humans are typically 4.9-11 mg/ml (see, e.g., Van Kessel, et al., Clin. Ex. Immunol., 1999, 118 (1): 102-107), thus the serum IgG1 levels of immunized Tg32-hFc mice are approach physiologically-relevant levels for humans, mimicking competition conditions for human FcRn-mediated IgG1 recycling.

Human or humanized monoclonal IgG1 antibodies in humans compete with endogenous serum IgG1 for hFcRn-mediated antibody recycling. The serum half-life of IgG1 was measured in Tg32-hFc mice to ensure that competition for hFcRn-mediated antibody recycling was occurring (see FIG. 3 of Low, B.E., et al. 2020). Tg32-hFc mice expressing chimeric IgG1 were injected with the humanized monoclonal IgG1 antibody trastuzumab (HERCEPTIN®) to examine competition for human FcRn recycling of IgG1 antibodies. Additionally, when injected with humanized mAbs, for example trastuzumab, this yielded a half-life of 8.8+/−0.6 days in naïve Tg32 mice, while transgenic expression of Fc humanized IgG1 in Tg32-hFc mice reduced half-life of trastuzumab to 7.4+/−0.2 days (naïve, p=0.06) and 6.5+/−0.3 days (immunized, p=0.01). “Naïve” refers to mice not previously contacted with an antigen (e.g., DNP-KLH), and “immunized” refers to mice previously contacted with an antigen (e.g., DNP-KLH). The serum half-life of trastuzumab in humans is 2-12 days, depending on dosage (see, e.g., “Trastuzumab”, DrugBank, drugbank.ca/drugs/DB00072). The decrease in serum half-life of the humanized trastuzumab antibody in Tg32-hFc mice expressing chimeric IgG1 suggests that there is increased competition for human FcRn-mediated recycling of IgG1, and that these mice closely recapitulate human FcRn-mediated antibody recycling.

### SEQUENCES

Mouse genomic region with chimeric human IgG1 (human region underlined-replaces mouse hinge, CH2 and CH3)

(SEQ ID NO: 1)
AACAAGAACAGGGGAAATCCTAGGGCTGACATTGCCAGTGGAAACATACAGGCTGGAGCTCTTT
AGTCAGGAGCTCCAGCTGTGATCTAGACATCAGGCAGGAAGATCAAATCTGTCCCAACAATACA
GGGGACAGAGGCTCAACCTAGAGTGTGAGCATCAGGGGCTGTGCAGGAGATTTCAGAGCTCAGG
TGCAGCAGAGACTAGCATGGCCCTGGGGATAAAGGGAAGGATCCAAGGGACAAGGGGATAATCC
TGGGGAGGTAAGGGCCAGCTTCGTGACAGAAGGTGGTGGTGTCCAACTTCAAGAGCCCTGTGCT
ACAATTTAAAAAAAAAAAAAAAGGAAAGGGACTTCTCTGTGTTTGGCAACACAAGTGCGATGCA
CAGGCAGGAAGATCAAATCTGTCCCAACAATACAGGGGACAGAGGGTCAACCTACAAAAGGAAA
GAACCTGGGGCAGTGTGAAGACAACACTGTAGAAGCCAAGGCTGAGTTCACTGAGCTCTCGTTA
GTGAGACTACACAGCAAGGAGGTGGCGGGCACTGAGCAGTGAGGCCCCGGGAAGTGGGGGTGAT
GGTGGTGACGGTGGTAACTGTTAAGAACTGGGGGAAAGAATTGTGGAGAACCAAGCTAAATAGT
TATGTCAAACCACATGTTTAGGAGCCTGGGTTGACTTCATAGGGAGTAGGCATGGAGGCTAATC
TAGAGGTTTGTGTATAGGCAAGAAGTGAATCCTGACCCAAGAATAGAGAGTGCTAAACGGACTT
AGTTCAAAGACAACTGAAAAAGACAATGCCTGCAAAACAAAGCTAAGGCCAGAGCTCTTGGACT
ATGAAGAGTTCAGGGAACCTAAGAACAGGGACCATCTGTGTACAGGCCAAGGCCGGTAGAAGCA
GCCTAGGAAGTGTCAAGAGCCAACGTGGCTGGGTGGGCAAAGACAGGAAGGGACTGTTAGGCTG
CAGGGATGTGCCGACTTCAATGTGCTTCAGTATTGTCCAGATTGTGTGCAGCCATATGGCCCAG
GTATAAGAGGTTTAACAGTGGAACACAGATGCCCACATCAGACAGCTGGGGGGGGGGGGTGAAC
ACAGATACCCATACTGGAAAGCAGGTGGGGCATTTTCCTAGGAACGGGACTGGGCTCAATGGCC
TCAGGTCTCATCTGGTCTGGTGATCCTGACATTGATAGGCCCAAATGTTGGATATCACCTACTC
CATGTAGAGAGTCGGGGACATGGGAAGGGTGCAAAAGAGCGGCCTTCTAGAAGGTTTGGTCCTG
TCCTGTCCTGTCTGACAGTGTAATCACATATACTTTTTCTTGTAGCCAAAACGACACCCCCATC
TGTCTATCCACTGGCCCCTGGATCTGCTGCCCAAACTAACTCCATGGTGACCCTGGGATGCCTG
GTCAAGGGCTATTTCCCTGAGCCAGTGACAGTGACCTGGAACTCTGGATCCCTGTCCAGCGGTG
TGCACACCTTCCCAGCTGTCCTGCAGTCTGACCTCTACACTCTGAGCAGCTCAGTGACTGTCCC
CTCCAGCACCTGGCCCAGCCAGACCGTCACCTGCAACGTTGCCCACCCGGCCAGCAGCACCAAG
GTGGACAAGAAAATTGGTGAGAGGACGTATAGGGAGGAGGGGTTCACTAGAGGTGAGGCTCAAG
CCATTAGCCTGCCTAAACCAACCAGGCTGGACAGCCATCACCAGGAAATGGATCTCAGCCCAGA
AGATCGAAAGTTGTTCTTCTCCCTTCTGGAGATTTCTATGTCCTTTACACTCATTGGTTAATAT
CCTGGGTTGGATTCCCACACATCTTGACAAACAGAGACAATTGAGTATCACCAGCCAAAAGTCA
TACCCAAAAACAGCCTGGCATGACCTCACACCAGACTCAAACTTACCCTACCTTTATCCTGGTG
GCTTCTCATCTCCAGACCCCAGTAACACATAGCTTTCTCTCCACAGTGCCCAAATCTTGTGACA
AAACTCACACATGCCCACCGTGCCCAGGTAAGCCAGCCCAGGCCTCGCCCTCCAGCTCAAGGCG
GGACAGGTGCCCTAGAGTAGCCTGCATCCAGGGACAGGCCCCAGCCGGGTGCTGACACGTCCAC
CTCCATCTCTTCCTCAGCACCTGAACTCCTGGGGGGACCGTCAGTCTTCCTCTTCCCCCCAAAA
CCCAAGGACACCCTCATGATCTCCCGGACCCCTGAGGTCACATGCGTGGTGGTGGACGTGAGCC
ACGAAGACCCTGAGGTCAAGTTCAACTGGTACGTGGACGGCGTGGAGGTGCATAATGCCAAGAC
AAAGCCGCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCAGCCCCCA
TCGAGAAAACCATCTCCAAAGCCAAAGGTGGGACCCGTGGGGTGCGAGGGCCACATGGACAGAG
GCCGGCTCGGCCCACCCTCTGCCCTGAGAGTGACCGCTGTACCAACCTCTGTCCCTACAGGGCA
GCCCCGAGAACCACAGGTGTACACCCTGCCCCCATCCCGGGATGAGCTGACCAAGAACCAGGTC
AGCCTGACCTGCCTGGTCAAAGGCTTCTATCCCAGCGACATCGCCGTGGAGTGGGAGAGCAATG
GGCAGCCGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATGCTCCGTG
ATGCATGAGGCTCTGCACAACCACTACACACAGAAGAGCCTCTCCCTGTCTCCTGGTAAATGAT
CCCAGTGTCCTTGGAGCCCTCTGGTCCTACAGGACTCTGACACCTACCTCCACCCCTCCCTGTG
TAAATAAAGCACCCAGCACTGCCTTGGGACCCTGCAATAATGTCCTGGTGATTTCTGAGATGTA
GAGTCTAGCTAGGTCATGGAATGAGGGGTCTCCATGGTTTGAGGCCTGAGTTGTGACTAAGGAA
AAACCCATAGGCCTACACTGCCACACCCAGCACTTTTGAATTTGCCTGACATGAAAAGAATTTA
CCTCTCCCTGGAAAGTGGAGCCTTATCCCTAGGCAGTTCCCTTACCAGACCTTCCTCTAGCTTG
CACTTTGTTCTGGGCACAGAATGTGTCTAACCCCCCAAAGCAAGGAAGACACAACCTCTACCTC
CCTCACTCTGTCCTTACCCCTTTTCCTGGCTAAGCATCTCACTGAGTGCGCTGAATAGATGCAT
GTGGCCACAGTCTTGCAGACAGACCCTTGCCATCTCTCCACTCAGCTTTCCAGAGGCTAAGTCT
AGCCCGTATGGTGATAATGCAGGGAGCTCTATGCTATCTCAGTGCTATCAGACTCCCAAGTGGA
GGATGAACATGGACCCATTAAAACCAACCTGCGCAGCAACACCCTGCCAATAAGGCCCGTATGT
GAAAATGTGCACACATCTACACATGCACAGGCACACACACACACACATGCATGGGCACACACAC
ATACAGAGAGAGAGAATCACAGAAACTCCCATGAGCATCCTATACAGTACTCAAAGATAAAAAG
GTACCAGGTCTACCCACATGATCATCCTCGGCATTTACAAGTGGGCCAACTGATACAGATAAAA
CTTTTCTATGCCAAGGACGCCAACATATACACAAGTCCGCTCATGACAAATCTGTCCCTGAACC
TCAGACTGGCGCCCGTGACTCATACAGTGGACACTCCTCCAAAGCTGTATAGCTTCCTTTACTT
CCCTGTGTGTACTTTCTCTGAAGTACACTCATCACACAGAAGAGGCCCTGTGATTACTCTGGCC
CTCTGTTCTTGGTCATCAGAGAATAGACAGAAGATCAGGCAAACTACACAGACACTTCCCACAA
TCATCACAGGCCCTGACTCTGCTCTCCAGTCTCAAAACTGAAGGCTGGAGCACACAGAATAAGC
TCCTGCACAGGCCAGGCCAGTATCGGGTCCAGTGTGTCTGACTGAGCCCAGGGACAAAATGGCA
GCACTTTGGGGAACTGAGGTTTCTGGTCCAAGAAGGAGAGATGGAGGCCCAGGGAGGGTCTGCT
GACCCAGCCCAGCCCAGCCCAGCTGCAGCTTTCTCCTGGGCCTCCATACAGCCTCCTGCCACAC
AGGGAATGGCCCTAGCCCCACCTTATTGGGACAAACACTGACCGCCCTCTCTGTCCAGGGCTGC
AACTGGACGAGACCTGTGCTGAGGCCCAGGACGGGGAGCTGGACGGGCTCTGGACGACCATCAC
CATCTTCATCAGCCTCTTCCTGCTCAGTGTGTGCTACAGCGCTGCTGTCACACTCTTCAAGGTC
AGCCATACTGTCCCCACAGTGTCTACAATGTCCTCATACTCTTCCCCATACTGTCCCTGTGGTG
ACCTATACCCCACACTGTCCCATGCTAATGACCACAGTCTTACATGCTATGTAATGCTGTCTAC
CCTTCTGTATGCACAGTCTCACAATGTCCCATGCAGTCTCCACGATGCTCCATACTGTCCCCAT
TCCAACCCATGCTGCCCCTTGTTCCCCGCTATGCTGTCCCATGCTATTGTCTGTATTTTCATGC
TCTTTTCACACTGTCCCTAGTGTCACATTCTGCCCATGTTGTCCACCACATTGTCCCCACTCTG
TACACAGCCTCACACTGTACCCTGCTACCCGATAATGTTCCCTGTTGTCCCCAACTCTCTCCCT
GCACCATTTGTCAACTGTCCCCTGAATTCCCATGTTGTTCCCACACTGTTAGTGTGTAATGTGC
TCTGTCCCA

### Human IgG1 Fc Hinge

(SEQ ID NO: 2)
PKSCDKTHTCP

### Human IgG1 Fc Heavy Chain

(SEQ ID NO: 3)
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGK

### gRNA-1

(SEQ ID NO: 4)
GCACAATCCCTGGGCACTGTGG

### gRNA-2

(SEQ ID NO: 5)
GAGCCTCTCCCACTCTCCTGG

chimeric IgG1 heavy chain, long variant, including human Fc hCH2, human hCH3, mouse CH1(human region underlined—replaces mouse hinge, CH2 and CH3)

(SEQ ID NO: 6)
KTTPPSVYPLAPGSAAQTNSMVTLGCLVKGYFPEPVTVTWNSGSLSSGV
HTFPAVLQSDLYTLSSSVTVPSSTWPSQTVTCNVAHPASSTKVDKKIVP
KSCDKTHTCPPCPAPELLGGPSVFLFPPKPKDTLMISRTPEVTCVVVDV
SHEDPEVKENWYVDGVEVHNAKTKPREEQYNSTYRVVSVLIVLHQDWLN
GKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVD
KSRWQQGNVFSCSVMHEALHNHYTQKSLSLSPGLQLDETCAEAQDGELD
GLWTTITIFISLFLLSVCYSAAVTLFKVKWIFSSVVELKQTLVPEYKNM
IGQAP

chimeric IgG1 heavy chain, short variant, including human Fc hCH2, human Fc hCH3, mouse CH1, (human regions underlined—replaces mouse hinge, CH2, and CH3)

(SEQ ID NO: 7)
KTTPPSVYPLAPGSAAQTNSMVTLGCLVKGYFPEPVTVTWNSGSLSSGV
HTFPAVLQSDLYTLSSSVTVPSSTWPSQTVTCNVAHPASSTKVDKKIVP
KSCDKTHTCPPCPAPELLGGPSVFLFPPKPKDTLMISRTPEVTCVVVDV
SHEDPEVKFNWYVDGVEVHNAKTKPREEQYNSTYRVVSVLIVLHQDWLN
GKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVD
KSRWQQGNVFSCSVMHEALHNHYTQKSLSLSPGK

What is claimed is:

1. A mouse comprising a nucleic acid encoding human neonatal Fc receptor (FCRN) and a nucleic acid encoding a chimeric immunoglobulin that comprises a human immunoglobulin gamma (IgG) Fc region and a mouse Fab region, wherein the mouse comprises a null mutation in endogenous FcRn.

2.-14. (canceled)

15. A nucleic acid or set of nucleic acids encoding a chimeric immunoglobulin that comprises a human immunoglobulin gamma (IgG) Fc region and a mouse Fab region.

16. A chimeric immunoglobulin comprising a human immunoglobulin gamma (IgG) Fc region and a mouse Fab region.

17. A method of producing a mouse comprising modifying the genome of the mouse to express a nucleic acid or set of nucleic acids encoding the chimeric immunoglobulin of claim 16.

18. A method of producing a mouse comprising modifying the genome of a B6.Cg-Fcgrt^{tm1Dcr}Tg(FCGRT)32Dcr/DcrJ mouse to express the nucleic acid or set of nucleic acids of claim 15.

19. A method comprising administering to the mouse of claim 1 a therapeutic antibody and assaying half-life of the therapeutic antibody in serum of the mouse.

20. The method of claim 19, wherein the therapeutic antibody is a monoclonal IgG antibody.

21. The method of claim 20, wherein the monoclonal IgG antibody is a monoclonal IgG1 antibody.

22. Use of the mouse of claim 1 to produce Fc-humanized IgG1 monoclonal antibodies.
